(19) United States
(12) Patent Application Publication
(10) Pub. No.: US 2024/0279696 A1
(43) Pub. Date: Aug. 22, 2024

(54) MICROORGANISM HAVING MODIFIED LACTOSE PERMEASE, AND METHOD FOR PRODUCING LACTOSE-CONTAINING OLIGOSACCHARIDE

(71) Applicant: KYOWA HAKKO BIO CO., LTD., Tokyo (JP)

(72) Inventors: Kenichiro TABATA, Tokyo (JP); Shinsuke UCHIYA, Tokyo (JP)

(73) Assignee: KYOWA HAKKO BIO CO., LTD., Tokyo (JP)

(21) Appl. No.: 17/785,813

(22) PCT Filed: Dec. 16, 2020
(86) PCT No.: PCT/JP2020/047049

§ 371 Date: Jun. 15, 2022

(30) Foreign Application Priority Data
Dec. 16, 2019 (JP) 2019-226278

(51) Int. Cl. C12P 19/00; C12N 9/12

(52) U.S. Cl. CPC C12P 19/00; C12N 9/1205; C12Y 207/01

(57) ABSTRACT

An object of the invention is to provide a method for producing a lactose-containing oligosaccharide through more efficient fermentative production. According to the invention, using a microorganism having an ability of producing a protein which is modified by replacing a specific amino acid residue with a different amino acid residue and which has lactose permease activity, a lactose-containing oligosaccharide such as 2′-fucosyllactose can be produced more efficiently as compared to the case using a microorganism having an ability of producing the wild-type protein having lactose permease activity.

### INCORPORATION-BY-REFERENCE OF MATERIAL ELECTRONICALLY SUBMITTED

Incorporated by reference in its entirety herein is a computer-readable nucleotide/amino acid sequence listing submitted concurrently herewith and identified as follows: 16,309 bytes ASCII (Text) file named “763449-ReplacementSequenceListing.txt,” created Nov. 17, 2022.

### TECHNICAL FIELD

The present invention relates to a method for producing a lactose-containing oligosaccharide which can produce a lactose-containing oligosaccharide highly efficiently.

### BACKGROUND ART

Milk oligosaccharides contained in human breast milk (HMOs) have been reported to have a phylactic effect against pathogenic bacteria or function as prebiotics and are receiving attention as an additive for infant formula due to the bioactivity (Non-Patent Literature 1).

So far, 130 kinds of HMO or more have been known, and most of them are lactose-containing oligosaccharides having a free lactose unit at the reducing terminal.

As a method for producing a lactose-containing oligosaccharide through fermentative production, a method of adding relatively inexpensive lactose as a substrate is known (Non-Patent Literature 2). Moreover, it is known that the uptake of lactose from outside of microbial cells is mediated by lactose permease (Non-Patent Literature 3).

However, a method for producing a lactose-containing oligosaccharide using a microorganism having a mutant lactose permease or the influence of a mutation of lactose permease on the productivity of a lactose-containing oligosaccharide is not known. At the same time, a more efficient method for producing a lactose-containing oligosaccharide is required due to the noteworthiness.

### CITATION LIST

### Non-Patent Literature

Non-Patent Literature 1: J Biotechnol (2016) 235:61-83
Non-Patent Literature 2: Curr Opin Biotechnol (2019) 56:130-137
Non-Patent Literature 3: J Biotechnol (2015) 210:107-115

### SUMMARY OF INVENTION

### Technical Problem

As described above, a more efficient method for producing a lactose-containing oligosaccharide is required. An object of the invention is to provide a method for producing a lactose-containing oligosaccharide through more efficient fermentative production.

### Solution to Problem

The invention relates to the followings.
1. A microorganism which has a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3] below and
   which has a higher ability of producing a lactose-containing oligosaccharide than a parent strain,
   [1] a protein consisting of the amino acid sequence represented by SEQ ID NO:2,
   [2] a mutant protein which is a protein consisting of an amino acid sequence having deletion, replacement, insertion and/or addition of 1 to 20 amino acids in the amino acid sequence represented by SEQ ID NO:2 and which has lactose permease activity, and
   [3] a homologous protein which is a protein consisting of an amino acid sequence having at least 80% identity with the amino acid sequence represented by SEQ ID NO:2 and which has lactose permease activity.
2. The microorganism according to the above 1 which has a protein consisting of an amino acid sequence containing replacement of the amino acid residue corresponding to position 319 of the amino acid sequence represented by SEQ ID NO:2 with L-aspartic acid or L-glutamic acid in the amino acid sequence of the protein described in any one of [1] to [3].
3. The microorganism according to the above 1 or 2 which has a protein consisting of an amino acid sequence containing replacement of the amino acid residue corresponding to position 319 of the amino acid sequence represented by SEQ ID NO:2 with L-glutamic acid in the amino acid sequence of the protein described in any one of [1] to [3].
4. The microorganism according to any one of the above 1 to 3 which is obtainable by transforming a parent strain with a recombinant DNA containing a DNA encoding a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3].
5. The microorganism according to the above 4 which is obtainable by incorporating the recombinant DNA containing a DNA encoding a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3] into the chromosome.
6. The microorganism according to any one of the above 1 to 5 which is obtainable by transforming a parent strain with a recombinant DNA containing a DNA having the nucleotide sequence represented by SEQ ID NO:3.
7. The microorganism according to the above 6 which is obtainable by incorporating the recombinant DNA containing a DNA having the nucleotide sequence represented by SEQ ID NO:3 into the chromosome.
8. The microorganism according to any one of the above 1 to 7, wherein the parent strain is a microorganism having an ability of producing a lactose-containing oligosaccharide.
9. A method for producing a lactose-containing oligosaccharide, comprising: culturing the microorganism according to any one of the above 1 to 8 in a medium and producing a lactose-containing oligosaccharide in a culture.
10. The production method according to the above 9, wherein the lactose-containing oligosaccharide is 2′-fucosyllactose.

### Advantageous Effects of Invention

According to the invention, a method for producing a lactose-containing oligosaccharide which can produce a lactose-containing oligosaccharide highly efficiently is provided.

### DESCRIPTION OF EMBODIMENTS

### 1. Microorganism of Invention

The microorganism of the invention is a microorganism having the protein described in (1) or (2) below and is a microorganism having a higher ability of producing a lactose-containing oligosaccharide than a parent strain.
(1) A protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3] below.
[1] A protein consisting of the amino acid sequence represented by SEQ ID NO:2.
[2] A mutant protein which is a protein consisting of an amino acid sequence having deletion, replacement, insertion and/or addition of 1 to 20 amino acids in the amino acid sequence represented by SEQ ID NO:2 and which has lactose permease activity.
[3] A homologous protein which is a protein consisting of an amino acid sequence having at least 80% identity with the amino acid sequence represented by SEQ ID NO:2 and which has lactose permease activity.
(2) The protein described in (1) above which consists of an amino acid sequence containing replacement of the amino acid residue corresponding to position 319 of the amino acid sequence represented by SEQ ID NO:2 with L-glutamic acid in the amino acid sequence of the protein described in any one of [1] to [3] above.

In the amino acid sequence of the protein as the origin described in any one of [1] to [3] above, the amino acid residue corresponding to position 319 of the amino acid sequence represented by SEQ ID NO:2 refers to the amino acid residue which is aligned at the same position as the amino acid residue at position 319 of the amino acid sequence of SEQ ID NO:2 when the amino acid sequence of the original protein and the amino acid sequence of SEQ ID NO:2 are aligned.

In the amino acid sequence of the protein described in (1) or (2) above, that the amino acid residue corresponding to position 319 of the amino acid sequence represented by SEQ ID NO:2 has been replaced with a different amino acid residue can be confirmed, for example, by aligning the amino acid sequence of the protein described in (1) or (2) above, for which the amino acid residue is to be confirmed, and the amino acid sequence of the original protein described in any one of [1] to [3] above.

An alignment of amino acid sequences can be created, for example, using known alignment program ClustalW [Nucelic Acids Research 22, 4673, (1994)]. ClustalW is available, for example, at http://www.ebi.ac.uk/clustalw/ (European Bioinformatics Institute). The parameters used for creating an alignment using ClustalW can be, for example, the default values.

The different amino acid residue described in (1) above may be either a natural or unnatural amino acid residue. Natural amino acids are L-alanine, L-asparagine, L-aspartic acid, L-glutamine, L-glutamic acid, glycine, L-histidine, L-isoleucine, L-leucine, L-lysine, L-arginine, L-methionine, L-phenylalanine, L-proline, L-serine, L-threonine, L-tryptophan, L-tyrosine, L-valine, L-cysteine and the like.

Examples of mutually replaceable amino acids are shown below. Amino acids included in a same group can be interchanged.
Group A: leucine, isoleucine, norleucine, valine, norvaline, alanine, 2-aminobutanoic acid, methionine, o-methylserine, t-butylglycine, t-butylalanine and cyclohexylalanine.
Group B: aspartic acid, glutamic acid, isoaspartic acid, isoglutamic acid, 2-aminoadipic acid and 2-aminosuberic acid.
Group C: asparagine and glutamine.
Group D: lysine, arginine, ornithine, 2,4-diaminobutanoic acid and 2,3-diaminopropionic acid.
Group E: proline, 3-hydroxyproline and 4-hydroxyproline.
Group F: serine, threonine and homoserine.
Group G: phenylalanine and tyrosine.

The different amino acid residue described in (1) above is preferably one selected from the amino acid residues listed in Group B above (aspartic acid, glutamic acid, isoaspartic acid, isoglutamic acid, 2-aminoadipic acid and 2-aminosuberic acid), and the amino acid residue is more preferably an L-form. The different amino acid residue described in (1) above is further preferably L-glutamic acid.

The mutant protein refers to a protein obtainable by artificially deleting or replacing an amino acid residue of the original protein or artificially inserting or adding an amino acid residue to the protein.

In the mutant protein, deletion, replacement, insertion or addition of an amino acid may be deletion, replacement, insertion or addition of 1 to 20 amino acids at any positions of a same sequence.

The deleted, replaced, inserted or added amino acids may be either natural or unnatural amino acids. Examples of the natural amino acids are the natural amino acids above.

Examples of interchangeable amino acids are as described above. Amino acids included in a same group can be interchanged.

Homologous proteins are proteins that an organism which exists in the nature has and refer to a group of proteins derived from proteins of the same evolutional origin. Homologous proteins have similar structures and functions. The identity of amino acid sequences or nucleotide sequences can be determined using the algorithm BLAST [Pro. Nat. Acad. Sci. USA, 90, 5873 (1993)] by Karlin and Altschul or FASTA [Methods Enzymol., 183, 63 (1990)]. Programs called BLASTN and BLASTX have been developed based on the algorithm BLAST [J. Mol. Biol., 215, 403 (1990)]. When nucleotide sequences are analyzed using BLASTN based on BLAST, the parameters are, for example, Score=100 and word length=12. When amino acid sequences are analyzed using BLASTX based on BLAST, the parameters are, for example, score=50 and word length=3. When the BLAST and Gap ped BLAST programs are used, the default parameters of the programs are used. Specific techniques for the analysis methods are known.

That the mutant protein or the homologous protein has lactose permease activity can be confirmed, for example, by the following method. First, a recombinant DNA having a DNA encoding the mutant protein or the homologous protein, for which the activity is to be confirmed, is produced by the following method. Next, a microorganism having no lactose permease activity, such as Escherichia coli strain W3110, which lacks lactose permease, is transformed with the recombinant DNA. In the end, the microorganism is cultured in a medium containing lactose as a saccharide source, and when the growth improves as compared with the parent strain, it is confirmed that the mutant protein or the homologous protein has lactose permease activity.

A specific example of the protein containing replacement of the amino acid residue corresponding to position 319 of the amino acid sequence represented by SEQ ID NO:2 with a different amino acid residue in the amino acid sequence of the protein described in (1) or (2) above is a protein consisting of the amino acid sequence represented by SEQ ID NO:32.

The parent strain refers to an original strain subjected to gene modification, transformation or the like. The original strain subjected to transformation by gene introduction is also called a host strain.

The parent strain of the microorganism of the invention may be any microorganism as long as the microorganism has an ability of producing a lactose-containing oligosaccharide.

The lactose-containing oligosaccharide refers to an oligosaccharide having a lactose unit at the reducing terminal. Examples of the lactose-containing oligosaccharide include 2′-fucosyllactose, 3-fucosyllactose, 2′,3-difucosyllactose, 3′-sialyllactose, 6′-sialyllactose, 3′-sialyl-3-fucosyllactose and the like.

As the microorganism having an ability of producing a lactose-containing oligosaccharide, a breeding strain to which an ability of producing a lactose-containing oligosaccharide has been artificially given or in which the ability has been enhanced can be preferably used.

Methods for artificially giving an ability of producing a lactose-containing oligosaccharide to the microorganism used as the parent strain or for enhancing the ability in the microorganism are: (a) a method of enhancing the expression of at least one enzyme involved in the biosynthetic pathway for generating the lactose-containing oligosaccharide from a sugar; (b) a method of increasing the number of copies of at least one gene encoding an enzyme involved in the biosynthetic pathway for generating the lactose-containing oligosaccharide from a sugar; (c) a method of relieving or canceling at least one mechanism of controlling the biosynthetic pathway for generating the lactose-containing oligosaccharide from a sugar; (d) a method of weakening or blocking at least one metabolic pathway branching from the biosynthetic pathway for generating the lactose-containing oligosaccharide from a sugar into a metabolite other than the target substance; and another method. The known methods can be used singly or in combination.

Specific examples of the enzyme involved in the biosynthetic pathway for generating the lactose-containing oligosaccharide from a sugar include known enzymes such as an enzyme having α1,2-fucosyltransferase activity for generating 2′-fucosyllactose using GDP-fucose and lactose as substrates, an enzyme having α1,3-fucosyltransferase activity for generating 3-fucosyllactose using GDP-fucose and lactose as substrates, an enzyme having α2,3-sialyltransferase activity for generating 3′-sialyllactose using CMP-sialic acid and lactose as substrates and an enzyme having α2,6-sialyltransferase activity for generating 6′-sialyllactose using CMP-sialic acid and lactose as substrates.

Specific examples of the method for giving or enhancing an ability of producing a lactose-containing oligosaccharide include known methods such as a method for enhancing the ability of producing 2′-fucosyllactose or 3-fucosyllactose by genetic engineering (Metabolic Engineering (2017) 41:23-38).

A breeding strain obtained by artificially giving an ability of supplying lactose as the precursor to a microorganism having an ability of producing a lactose-containing oligosaccharide or by enhancing the ability in the microorganism can also be used.

Methods for artificially giving an ability of supplying lactose from a sugar to the microorganism used as the parent strain or for enhancing the ability in the microorganism are: (a) a method of relieving or canceling at least one mechanism of controlling the biosynthetic pathway for generating lactose from a sugar; (b) a method of enhancing the expression of at least one enzyme involved in the biosynthetic pathway for generating lactose from a sugar; (c) a method of increasing the number of copies of at least one gene encoding an enzyme involved in the biosynthetic pathway for generating lactose from a sugar; (d) a method of relieving or canceling at least one mechanism of decomposing lactose; (e) a method of weakening or blocking at least one metabolic pathway branching from the biosynthetic pathway for generating lactose from a sugar into a metabolite other than the target substance; and another method. The known methods can be used singly or in combination.

Specific examples of the enzyme involved in the biosynthetic pathway for generating lactose from a sugar include known enzymes such as an enzyme having lactose synthase activity for generating lactose using glucose and UDP-galactose as substrates.

Specific examples of the method for giving or enhancing the ability of supplying lactose include known methods such as a method of decreasing or inactivating β-galactosidase activity involved in decomposition of lactose (Metabolic Engineering (2017)
41:23-38).

The microorganism having an ability of producing a lactose-containing oligosaccharide may be any microorganism but is preferably a prokaryote or a yeast strain, more preferably a prokaryote belonging to the genus Escherichia, the genus Serratia, the genus Bacillus, the genus Brevibacterium, the genus Corynebacterium, the genus Microbacterium, the genus Pseudomonas or the like or a yeast strain belonging to the genus Saccharomyces, the genus Schizosaccharomyces, the genus Kluyveromyces, the genus Trichosporon, the genus Schwanniomyces, the genus Pichia, the genus Candida or the like, most preferably a prokaryote such as Escherichia coli BL21 codon plus, Escherichia coli XL1-Blue, Escherichia coli XL2-Blue (all manufactured by Agilent Technologies, Ltd.), Escherichia coli BL21(DE3)pLysS (manufactured by Merck Millipore), Escherichia coli DH5α, Escherichia coli HST08 Premium, Escherichia coli HST02, Escherichia coli HST04 dam/dcm, Escherichia coli JM109, Escherichia coli HB101, Escherichia coli CJ236, Escherichia coli BNM71-18 mutS, Escherichia coli MV1184, Escherichia coli TH2 (all manufactured by Takara Bio Inc.), Escherichia coli W, Escherichia coli JM101, Escherichia coli W3110, Escherichia coli W3110S (Kyow34, National BioResource Project), Escherichia coli MG1655, Escherichia coli DH1, Escherichia coli MC1000, Escherichia coli W1485, Escherichia coli MP347, Escherichia coli NM522, Escherichia coli ATCC9637, Escherichia coli KY3591 (deposit number: NITE BP-03062), Serratiaficaria, Serratiafonticola, Serratia liquefaciens, Serratia marcescens, Bacillus subtilis, Bacillus amyloliquefaciens, Brevibacterium immariophilum ATCC14068, Brevibacterium saccharolyticum ATCC14066, Corynebacterium ammoniagenes, Corynebacterium glutamicum ATCC13032, Corynebacterium glutamicum ATCC14067, Corynebacterium glutamicum ATCC13869, Corynebacterium acetoacidophilum ATCC13870, Microbacterium ammoniaphilum ATCC15354 and Pseudomonas sp. D-0110 or a yeast strain such as Saccharomyces cerevisiae, Schizosaccharomyces pombe, Kluyveromyces lactis, Trichosporon pullulans, Schwanniomyces alluvius, Pichia pastoris and Candida utilis.

Escherichia coli KY3591 has been deposited to NITE Patent Microorganisms Depositary (NPMD), National Institute of Technology and Evaluation (NITE) located at #122, 2-5-8 Kazusakamatari, Kisarazu-shi, Chiba, Japan (postal code 292-0818). The receipt date (deposit date) is Nov. 18, 2019, and the accession number is NITE BP-03062.

A microorganism obtained by later giving an ability of producing a lactose-containing oligosaccharide to a microorganism having the protein of (1) or (2) above produced using the host strain or by enhancing the ability in the microorganism and a microorganism obtained by further giving an ability of supplying lactose to the microorganism or by enhancing the ability in the microorganism which are microorganisms having a higher ability of producing a lactose-containing oligosaccharide than the parent strain are also the microorganisms of the invention.

Examples of the microorganism having the protein described in (1) or (2) above include a microorganism obtained by transforming a parent strain with a recombinant DNA having the DNA described in any one of (3) to (6) below and a microorganism obtained by incorporating the recombinant DNA into the chromosome.
(3) A DNA encoding the protein described in (1) or (2) above.
(4) A DNA encoding a protein consisting of an amino acid sequence containing replacement of the amino acid residue corresponding to position 319 of the amino acid sequence represented by SEQ ID NO:2 with a different amino acid residue in the amino acid sequence of a protein encoded by the DNA described in any one of [4] to [6] below.
[4] A DNA having the nucleotide sequence represented by SEQ ID NO:1.
[5] A DNA which hybridizes with a DNA consisting of a nucleotide sequence complementary to the nucleotide sequence represented by SEQ ID NO:1 under stringent conditions and which encodes a homologous protein having lactose permease activity.
[6] A DNA which consists of a nucleotide sequence having at least 95%, preferably at least 97%, further preferably at least 98%, most preferably at least 99% identity with the nucleotide sequence represented by SEQ ID NO:1 and which encodes a homologous protein having lactose permease activity.
(5) The DNA described in (4) above which encodes a protein consisting of an amino acid sequence containing replacement of the amino acid residue corresponding to position 319 of the amino acid sequence represented by SEQ ID NO:2 with L-glutamic acid in the encoded amino acid sequence.
(6) A DNA having the nucleotide sequence represented by SEQ ID NO:3.

To hybridize used above is a step in which a DNA hybridizes with a DNA having a specific nucleotide sequence or a part of the DNA. Therefore, the nucleotide sequence of the DNA which hybridizes with the DNA having a specific nucleotide sequence or a part of the DNA may be a DNA having a length which is useful as a probe for Northern or Southern blotting analysis or which can be used as an oligonucleotide primer for PCR analysis.

An example of the DNA used as a probe is a DNA of at least 100 bases or more, preferably 200 bases or more, more preferably 500 bases or more. An example of the DNA used as a primer is a DNA of at least 10 bases or more, preferably 15 bases or more.

A method for DNA hybridization experiment is well known, and the experiment can be performed by, for example, determining the conditions for hybridization according to Molecular Cloning, 4th edition (Cold Spring Harbor Laboratory Press (2012)), Methods for General and Molecular Bacteriology (ASM Press (1994)) and Immunology methods manual (Academic press (1997)) as well as many other standard textbooks.

Further, also according to an instructional manual accompanying a commercially available hybridization kit, a DNA which hybridizes under stringent conditions can be obtained. An example of the commercially available hybridization kit is random primed DNA labeling kit (manufactured by Roche Diagnostics GmbH), in which a probe is produced by the random prime method and in which hybridization is performed under stringent conditions.

The stringent conditions are, for example, conditions in which a filter on which a DNA has been immobilized and a probe DNA are incubated overnight at 42° C. in a solution containing 50% formamide, 5×SSC (750 mM sodium chloride and 75 mM sodium citrate), 50 mM sodium phosphate (pH 7.6), 5×Denhardt's solution, 10% dextran sulfate and 20 μg/L denatured salmon sperm DNA and in which the filter is then washed, for example, in a 0.2×SSC solution at about 65° C.

The various conditions can also be set by adding or changing a blocking reagent used for suppressing the background in the hybridization experiment. The addition of the blocking reagent may be accompanied by a change in hybridization conditions for adapting the conditions.

An example of the DNA which can hybridize under the stringent conditions is a DNA consisting of a nucleotide sequence having at least 95%, preferably at least 97%, further preferably at least 98%, most preferably at least 99% identity with the nucleotide sequence represented by SEQ ID NO:1 when performing calculation based on the parameters using the program such as BLAST or FASTA described above.

The microorganism obtained by transforming a parent strain with a recombinant DNA having the DNA described in any one of (3) to (6) above and the microorganism obtained by incorporating the recombinant DNA into the chromosome can be created by the following methods.

The DNA described in any one of (3) to (6) above can be obtained, for example, using a DNA encoding a protein consisting of the amino acid sequence represented by SEQ ID NO:2, by introducing a mutation into the nucleotide sequence of the part encoding the amino acid residue corresponding to position 319 of the amino acid sequence represented by SEQ ID NO:2 located on the DNA by a site-specific mutagenesis method described, for example, in Molecular Cloning, 4th edition (Cold Spring Harbor Laboratory Press (2012)), Current Protocols in Molecular Biology (JOHN WILEY & SONS, INC.) or the like and thus replacing the nucleotide sequence with a nucleotide sequence encoding any amino acid residue. Alternatively, the DNA of the invention can be obtained also using PrimeSTAR Mutagenesis Basal Kit (manufactured by Takara Bio Inc.).

The DNA can also be obtained by a similar method using a DNA encoding a mutant protein which consists of an amino acid sequence having deletion, replacement, insertion and/or addition of 1 to 20 amino acids in the amino acid sequence represented by SEQ ID NO:2 and which has lactose permease activity, by introducing a mutation into the nucleotide sequence of the part, in the amino acid sequence of the mutant protein, encoding the amino acid residue which corresponds to position 319 of the amino acid sequence represented by SEQ ID NO:2 when the amino acid sequence represented by SEQ ID NO:2 and the amino acid sequence of the mutant protein are aligned by the above method.

The DNA can also be obtained using a DNA encoding a homologous protein which has an amino acid sequence having at least 80% identity with the amino acid sequence represented by SEQ ID NO:2 and which has lactose permease activity, by introducing a mutation into the nucleotide sequence of the part, in the amino acid sequence of the homologous protein, encoding the amino acid residue which corresponds to position 319 of the amino acid sequence represented by SEQ ID NO:2 when the amino acid sequence of the homologous protein and the amino acid sequence represented by SEQ ID NO:2 are aligned by the above method.

The DNA encoding a protein consisting of the amino acid sequence represented by SEQ ID NO:2 can be obtained, for example, by Southern hybridization for a chromosomal DNA library of a microorganism, preferably the genus Escherichia, more preferably Escherichia coli strain W3110 using a probe which can be designed based on the nucleotide sequence of the DNA encoding a protein consisting of the amino acid sequence represented by SEQ ID NO:2 or by PCR [PCR Protocols, Academic Press (1990)] using primer DNAs which can be designed based on the DNA encoding a protein consisting of the amino acid sequence represented by SEQ ID NO:2 and using the chromosomal DNA of Escherichia coli strain W3110 as a template.

Escherichia coli strain W3110 (ATCC27325) can be acquired from American Type Culture Collection (ATCC).

An example of the DNA encoding a protein consisting of the amino acid sequence represented by SEQ ID NO:2 is a DNA consisting of the nucleotide sequence represented by SEQ ID NO:1.

The DNA encoding the mutant protein which consists of an amino acid sequence having deletion, replacement, insertion and/or addition of 1 to 20 amino acids in the amino acid sequence represented by SEQ ID NO:2 and which has lactose permease activity described in [2] above can be obtained, for example, by subjecting a DNA consisting of the nucleotide sequence represented by SEQ ID NO:1 as a template to Error-Prone PCR or the like.

Alternatively, also by PCR using a set of PCR primers having nucleotide sequences designed to introduce the target mutation (deletion, replacement, insertion or addition) at the 5′ ends [Gene, 77, 51 (1989)], the DNA encoding the mutant protein which has an amino acid sequence having deletion, replacement, insertion or addition of 1 to 20 amino acids in the amino acid sequence represented by SEQ ID NO:2 and which has lactose permease activity of [2] above can be obtained.

Further, the DNA can be obtained also according to an instructional manual accompanying a commercially available site-specific mutagenesis kit. An example of the commercially available site-specific mutagenesis kit is PrimeSTAR (registered trademark) Mutagenesis Basal Kit (manufactured by Takara Bio Inc.), which can introduce a mutation (deletion, replacement, insertion or addition) to the site to which the target mutation is to be introduced.

That is, first, a set of mutagenesis primers in which the 15 bases at the 5′ ends overlap each other are designed using a plasmid having a nucleotide sequence designed to introduce the target mutation (deletion, replacement, insertion or addition) as a template. At this point, the overlapped part includes the target mutation. Next, PCR is performed using the mutagenesis primers and using a plasmid having a nucleotide sequence to which the target mutation is to be introduced as a template. When Escherichia coli is transformed with the amplified fragment thus obtained, a plasmid having the nucleotide sequence having the target mutation can be obtained.

The DNA encoding the homologous protein which has an amino acid sequence having at least 80% identity with the amino acid sequence represented by SEQ ID NO:2 and which has lactose permease activity described in [3] above can be obtained by, for example, searching various gene sequence databases for a nucleotide sequence having at least 80%, preferably at least 90%, more preferably at least 95%, most preferably at least 99% identity with the nucleotide sequence represented by SEQ ID NO:2 and performing a similar method to the method for obtaining the DNA encoding a protein consisting of the amino acid sequence represented by SEQ ID NO:2 using a probe DNA or primer DNAs which can be designed based on the nucleotide sequence or the amino acid sequence obtained by the search and using a microorganism having the DNA. The identity of nucleotide sequences or amino acid sequences can be determined by a similar method to that described above.

An example of the recombinant DNA having the DNA described in any one of (3) to (6) above is a recombinant DNA that is a DNA in which the DNA can autonomously replicate in the parent strain and that has the DNA described in any one or more of (3) to (6) above incorporated in an expression vector including a promoter at a position allowing the transcription of the DNA described in any one of (3) to (6) above.

The recombinant DNA described in any one of (3) to (6) above which is a DNA that can be incorporated into the chromosome in the parent strain is also the recombinant DNA having the DNA described in any one of (3) to (6) above.

When the recombinant DNA is a recombinant DNA which can be incorporated into the chromosome, a promoter does not have to be included.

When a prokaryote such as a bacterium is used as the host strain, the recombinant DNA which can autonomously replicate in the parent strain is preferably a recombinant DNA composed of a promoter, a ribosome binding sequence, the DNA described in any one or more of (3) to (6) above and a terminator sequence. A gene which controls the promoter may be included.

The distance between the Shine-Dalgarno sequence, which is a ribosome binding sequence, and the start codon is preferably adjusted to an appropriate distance, for example, to 6 to 18 bases.

In the recombinant DNA which can autonomously replicate in the parent strain, a terminator sequence is not necessarily required for the expression of the DNA, but a terminator sequence is preferably located just below the structural gene.

When a microorganism belonging to the genus Escherichia is used as the parent strain, examples of the expression vector include pColdI, pSTV28, pUC118 (all manufactured by Takara Bio Inc.), pET21a, pCDF-1b, pRSF-1b (all manufactured by Merck Millipore), pMAL-c5× (manufactured by New England Biolabs Inc.), pGEX-4T-1, pTrc99A (both manufactured by GE Healthcare Bio-Sciences), pTrcHis, pSE280 (both manufactured by Thermo Fisher Scientific), pGEMEX-1 (manufactured by Promega Corporation), pQE-30, pQE-60, pQE80L (all manufactured by Qiagen), pET-3, pBluescriptII SK(+), pBluescriptII KS(−) (all manufactured by Agilent Technologies, Ltd.), pKYP10 (JP-A-S58-110600), pKYP200 [Agric. Biol. Chem., 48, 669 (1984)], pLSAl [Agric. Biol. Chem., 53, 277 (1989)], pGEL1 [Proc. Natl. Acad. Sci., USA, 82, 4306 (1985)], pTrS30 [prepared from Escherichia coli JM109/pTrS30 (FERM BP-5407)], pTrS32 [prepared from Escherichia coli JM109/pTrS32 (FERM BP-5408)], pTK31 [APPLIED AND ENVIRONMENTAL MICROBIOLOGY, 2007, Vol. 73, No. 20, p 6378-6385], pPE167 (Appl. Environ. Microbiol. 2007, 73:6378-6385), pPAC31 (WO98/12343), pUC19 [Gene, 33, 103 (1985)], pPA1 (JP-A-S63-233798) and the like.

When the expression vectors are used, the promoter may be any promoter which functions in the cells of the microorganism belonging to the genus Escherichia, but for example, a promoter derived from Escherichia coli, a phage or the like, such as trp promoter, gapA promoter, lac promoter, PL promoter, PR promoter and PSE promoter, can be used. Moreover, an artificially designed and modified promoter, such as a promoter in which two trp promoters are tandemly linked, tac promoter, trc promoter, IacT5 promoter, IacT7 promoter and let I promoter, can also be used.

When a coryneform bacterium is used as the parent strain, examples of the expression vector include pCG1 (JP-A-S57-134500), pCG2 (JP-A-S58-35197), pCG4 (JP-A-557-183799), pCG11 (JP-A-S57-134500), pCG116, pCE54, pCB101 (all in JP-A-S58-105999), pCE51, pCE52, pCE53 [all in Molecular and General Genetics, 196, 175 (1984)] and the like.

When the expression vectors are used, the promoter may be any promoter which functions in the cells of the coryneform bacterium, but for example, P54-6 promoter [Appl. Microbiol. Biotechnol., 53, p⁶⁷⁴-6⁷⁹ (2000)] can be used.

When a yeast strain is used as the parent strain, examples of the expression vector include YEp13 (ATCC37115), YEp24 (ATCC37051), YCp50 (ATCC37419), pHS19, pHS15 and the like.

When the expression vectors are used, the promoter may be any promoter which functions in the cells of the yeast strain, but examples thereof include promoters such as PHOS promoter, PGK promoter, GAP promoter, ADH promoter, gall promoter, gal10 promoter, heat shock polypeptide promoter, MFα1 promoter and CUP1 promoter.

The microorganism obtained by transforming a parent strain with the recombinant DNA refers to a microorganism which can produce the protein encoded by the DNA through transcription of the DNA as a result of introduction of the recombinant DNA as a plasmid capable of autonomously replicating in the parent strain or incorporation of the recombinant DNA into the chromosome of the parent strain.

An example of the method for confirming that the DNA described in (3) to (6) above is transcribed and that the protein encoded by the DNA is produced is measurement of the transcription amount of the DNA by Northern blotting or of the production amount of the protein by Western blotting.

The nucleotide sequence of the obtained DNA described in (3) to (6) above can be determined by incorporating the DNA into a vector by a general method directly or after cleavage with an appropriate restriction enzyme or the like, introducing the obtained recombinant DNA into host cells and then analyzing by a generally used nucleotide sequence analysis method such as the dideoxy method [Proc. Nat. Acad. Sci., USA, 74, 5463 (1977)] or using a nucleotide sequence analyzer such as Applied Biosystems 3500 genetic analyzer and Applied Biosystems 3730DNA analyzer (both manufactured by Thermo Fisher Scientific).

Examples of the host cells which can be used for determining the nucleotide sequence of the DNA of the invention include Escherichia coli DH5a, Escherichia coli HST08 Premium, Escherichia coli HST02, Escherichia coli HST04 dam-/dcm-, Escherichia coli JM109, Escherichia coli HB101, Escherichia coli CJ236, Escherichia coli BMH71-18 mutS, Escherichia coli MV1184, Escherichia coli TH2 (all manufactured by Takara Bio Inc.), Escherichia coli XL1-Blue, Escherichia coli XL2-Blue (both manufactured by Agilent Technologies, Ltd.), Escherichia coli DH1, Escherichia coli MC1000, Escherichia coli W1485, Escherichia coli W3110, Escherichia coli MP347, Escherichia coli NM522 and the like.

The vector is pBluescriptII KS(+), pPCR-Script Amp SK(+) (both manufactured by Agilent Technologies, Ltd.), pT7Blue (manufactured by Merck Millipore), pCRII (manufactured by Thermo Fisher Scientific), pCR-TRAP (manufactured by GenHunter Corporation), pDIRECT [Nucleic Acids Res., 18, 6069 (1990)] or the like.

As the method for introducing the recombinant DNA, any method for introducing a DNA into host cells can be used, and examples thereof include the method using calcium ions [Proc. Natl. Acad. Sci., USA, 69, 2110 (1972)], the protoplast method (JP-A-S63-248394), the electroporation method [Nucleic Acids Res., 16, 6127 (1988)] and the like.

When the obtained DNA is a partial length DNA as a result of the nucleotide sequence determination, the full-length DNA can be obtained by Southern hybridization method for a chromosomal DNA library using the partial length DNA as a probe or the like.

Furthermore, the target DNA can also be prepared by chemical synthesis based on the determined nucleotide sequence of the DNA using 8905-type DNA synthesizer manufactured by PerSeptive Biosystems, Inc. or the like.

Here, by substituting a base of the nucleotide sequence of the DNA described in (3) to (6) above to achieve an optimum codon for the expression in the parent strain, the expression level of the protein encoded by the DNA can also be increased. The information on the codon usage frequency of the host cells can be obtained through public database.

By inserting the DNA fragment prepared in the above manner in the downstream of the promoter of the appropriate expression vector, the recombinant DNA that the microorganism of the invention has can be produced.

An example of such a recombinant DNA is pYHA2, which is described below in the Examples.

Examples of the method for introducing the recombinant DNA as a plasmid capable of autonomously replicating in the parent strain include methods such as the method using calcium ions, the protoplast method and the electroporation method.

An example of the method for incorporating the recombinant DNA into the chromosome of the parent strain is a homologous recombination method. An example of the homologous recombination method is a method using a homologous recombination plasmid which can be produced by linking to a plasmid DNA having a drug-resistant gene which cannot autonomously replicate in the host cells for introduction. Moreover, an example of the method using homologous recombination which is often used for Escherichia coli is a method for introducing a recombinant DNA using the homologous recombination system of lambda phage [Proc. Natl. Acad. Sci. USA, 97, 6641-6645 (2000)].

Furthermore, using a selection method using the phenomenon that Escherichia coli becomes sucrose-sensitive due to levansucrase of Bacillus subtilis incorporated into the chromosome together with the recombinant DNA, a selection method using the phenomenon that Escherichia coli becomes streptomycin-sensitive by incorporation of wild-type rpsL gene into Escherichia coli having streptomycin-resistant mutant rpsL gene [Mol. Microbiol., 55, 137 (2005), Biosci. Biotechnol. Biochem., 71, 2905 (2007)] or the like, Escherichia coli in which the target region on the chromosomal DNA of the host cells has been replaced with the recombinant DNA can be obtained.

That the microorganism created by the above method is a microorganism having a higher ability of producing a lactose-containing oligosaccharide than the parent strain can be confirmed, for example, by culturing the parent strain and the created microorganism in a medium and comparing the production amounts of the lactose-containing oligosaccharide. An example of such a microorganism is strain KFL/pYHA2, which is described below in the Examples.

### 2. Production Method of Fucose-Containing Oligosaccharide of Invention

The method for producing a fucose-containing oligosaccharide of the invention is a method for producing a fucose-containing oligosaccharide characterized by culturing the microorganism of 1 above in a medium and producing a fucose-containing oligosaccharide in a culture.

The method for culturing the microorganism of 1 above can be conducted according to a general method used for culturing a microorganism. As the medium for culturing the microorganism, both a natural medium and a synthetic medium may be used as long as the medium contains a carbon source, a nitrogen source, an inorganic salt or the like which the microorganism can assimilate and as long as the microorganism can be cultured efficiently in the medium.

The carbon source may be a carbon source which the microorganism can assimilate, and for example, sugars such as glucose, fructose, sucrose, molasses containing such sugar, starch and starch hydrolysate, organic acids such as acetic acid and propionic acid, alcohols such as glycerol, ethanol and propanol and the like can be used.

As the nitrogen source, for example, ammonia, ammonium salts of inorganic acids or organic acids such as ammonium chloride, ammonium sulfate, ammonium acetate and ammonium phosphate and other nitrogen-containing compounds as well as peptone, meat extract, yeast extract, corn steep liquor, casein hydrolysate, soybean meal, soybean meal hydrolysate, fermentation bacteria, digested products thereof and the like can be used.

Examples of the inorganic salt include monopotassium phosphate, dipotassium phosphate, magnesium phosphate, magnesium sulfate, sodium chloride, ferrous sulfate, manganese sulfate, copper sulfate, calcium carbonate and the like.

The culturing is generally performed preferably under aerobic conditions such as shaking culture and submerged spinner culture under aeration. The culture temperature is generally 15 to 40° C., and the culture period is generally 5 hours to 7 days. The pH of the culture solution during the culturing is generally maintained at 3.0 to 9.0. The pH is adjusted using an inorganic or organic acid, an alkaline solution, urea, calcium carbonate, ammonia or the like.

An antibiotic such as ampicillin and tetracycline may be added to the medium during the culturing according to the need. When a microorganism transformed with an expression vector using an inducible promoter as the promoter is cultured, an inducer may be added to the medium according to the need. For example, isopropyl-β-D-thiogalactopyranoside (IPTG) or the like may be added to the medium when a microorganism transformed with an expression vector using lac promoter is cultured, and indoleacrylic acid or the like may be added to the medium when a microorganism transformed with an expression vector using trp promoter is cultured.

During the culturing, a precursor required for the production of a lactose-containing oligosaccharide, such as lactose and N-acetyllactosamine, may be added to the medium according to the need.

By producing and accumulating a lactose-containing oligosaccharide in the culture through the culturing and collecting the lactose-containing oligosaccharide from the culture, a lactose-containing oligosaccharide can be produced.

The produced lactose-containing oligosaccharide can be analyzed by a general method using carbohydrate ion chromatography or the like. The lactose-containing oligosaccharide can be collected from the culture or a treated product of the culture by a general method using activated carbon, an ion exchange resin or the like. When the lactose-containing oligosaccharide accumulates in bacterial cells, the lactose-containing oligosaccharide can be collected, for example, by crushing the bacterial cells by ultrasonic waves or the like, removing the bacterial cells by centrifugation and collecting the lactose-containing oligosaccharide from the obtained supernatant using activated carbon, an ion exchange rein or the like.

### Analysis Examples

In the Examples, 2′-fucosyllactose was analyzed and quantified by the procedures shown below. A culture solution containing a microorganism after culturing was centrifuged, and the supernatant was collected. 2′-Fucosyllactose contained in the supernatant was analyzed with sugar analyzer ICS-5000 (manufactured by Thermo Fisher Scientific).

### Analysis Conditions

Column: CarboPAC PA1
Column temperature: 25° C.
Mobile phases: (mobile phase A) water
(mobile phase B) 500 mmol/L sodium hydroxide
(mobile phase C) 300 mmol/L sodium acetate
Mixing ratio of mobile phase A, mobile phase B and mobile phase C:
(0 to 10 minute) gradient of 80:20:0 to 70:20:10
(10 to 18 minute) 70:20:10
(18 to 25 minute) 80:20:0
Flow rate: 0.8 mL/min
Detector: pulsed amperometric detector

### EXAMPLES

Details of the Examples are shown below, but the invention is not limited to the Examples.

### [Example 1] Creation of Microorganism Used for Producing 2′-Fucosyllactose

### (1) Acquisition of DNA Fragment Used as Marker for Gene Deletion

PCR was performed using DNAs consisting of the nucleotide sequences shown in “Primer Set” in Table 1 as primer sets and using the DNAs shown in “Template” in Table 1 as templates, and amplified DNA fragments were obtained.

TABLE 1

| Primer Set | | Amplified DNA |
|---|---|---|
| (SEQ ID NOs:) | Template | Fragment |
| 3 and 4 | pHSG396 (manufactured by Takara Bio Inc.) | cat |
| 5 and 6 | Genomic DNA of Bacillus subtilis strain 168 | sacB |

The genomic DNA of Bacillus subtilis strain 168 was prepared by a general method. The amplified DNA fragment, cat, includes the region from about 200 bp upstream of cat gene on pHSG396 to about 50 bp downstream thereof. The amplified DNA fragment, sacB, includes the region from about 300 bp upstream of sacB gene on the genomic DNA of Bacillus subtilis strain 168 to about 100 bp downstream thereof.

Next, PCR was performed using the amplified DNA fragments, cat and sacB, as templates and using DNAs consisting of the nucleotide sequences represented by SEQ ID NOs:3 and 6 as a primer set, and a DNA fragment including cat gene and sacB gene (called cat-sacB below) was obtained.

(2) Creation of Escherichia coli which has Lost β-Galactosidase Activity, Lactose Permease Activity and Colanic Acid-Producing Activity

Escherichia coli in which the DNA encoding β-galactosidase (called lacZ gene below), the DNA encoding lactose permease (called lacY gene below) and the DNAs encoding colanic acid production-related proteins (called wcaJ, wzxC, wcaK, wcaL or wcaM gene below) were deleted was created by the following method. Here, lacZ and lacY (called lacZY below) and wcaJ, wzxC, wcaK, wcaL and wcaM (called wcaJ-wzxC-wcaKLM below) each form an operon on the Escherichia coli genome.

PCR was performed using the genomic DNA of Escherichia coli strain KY3591 prepared by a general method as a template and using DNAs consisting of the nucleotide sequences shown in “Primer Set” in Table 2 as primer sets, and amplified DNA fragments were obtained.

TABLE 2

| Primer Set | Amplified DNA | |
|---|---|---|
| (SEQ ID NOs:) | Fragment | Remarks |
| 7 and 8 | lacZ upstream 1 | The sequences at the 5′ ends of the nucleotide |
| | | sequences represented by SEQ ID NOs: 3 and |
| | | 7 are complementary to each other. |
| 9 and 10 | lacY downstream 1 | The sequences at the 5′ ends of the nucleotide |
| | | sequences represented by SEQ ID NOs: 6 and |
| | | 9 are complementary to each other. |
| 8 and 11 | lacZ upstream 2 | The sequences at the 5′ ends of the nucleotide |
| 10 and 12 | lacY downstream 2 | sequences represented by SEQ ID NOs: 11 |
| | | and 12 are complementary to each other. |

Here, lacZ upstream 1 and lacZ upstream 2 include the region from the start codon of lacZ gene to about 900 bp upstream thereof. Moreover, lacY downstream 1 and lacY downstream 2 include the region from the stop codon of lacY gene to about 800 bp downstream thereof.

PCR was performed using a mixture containing lacZ upstream 1, lacY downstream 1 and cat-sacB fragment at equivalent molar ratios as a template and using DNAs consisting of the nucleotide sequences represented by SEQ ID NOs:8 and 10 as a primer set, and a DNA fragment consisting of a sequence in which cat-sacB fragment was inserted into the sequence of a region around lacZ and lacY genes (called lacZY::cat-sacB below) was obtained.

PCR was performed using a mixture containing lacZ upstream 2 and lacY downstream 2 at equivalent molar ratios as a template and using DNAs consisting of the nucleotide sequences represented by SEQ ID NOs:8 and 10 as a primer set, and a DNA fragment consisting of a sequence which did not include lacZ or lacY and in which the upstream of lacZ and the downstream of lacY were linked directly (called ΔlacZY below) was obtained.

By introducing lacZY::cat-sacB fragment into Escherichia coli strain W3110 having plasmid pKD46 including a gene encoding λ recombinase [Datsenko, K. A., Warner, B. L., Proc. Natl. Acad. Sci., USA, Vol. 97, 6640-6645 (2000)] by the electroporation method, a transformant exhibiting resistance to chloramphenicol and sensitivity to sucrose (a transformant in which lacZ and lacY genes were replaced with lacZY::cat-sacB) was obtained.

By introducing ΔlacZY fragment into the transformant by the electroporation method, transformants exhibiting sensitivity to chloramphenicol and resistance to sucrose (transformants in which lacZY::cat-sacB was replaced with ΔlacZY) were obtained. A transformant exhibiting sensitivity to ampicillin (a transformant lacking pKD46) was further obtained from the transformants. The transformant was named W3110ΔlacZY.

In a similar manner, PCR was performed using the genomic DNA of Escherichia coli strain KY3591 (accession number is NITE BP-03062) as a template and using DNAs consisting of the nucleotide sequences shown in “Primer Set” in Table 3 as primer sets, and amplified DNA fragments were obtained.

TABLE 3

| Primer Set | Amplified DNA | |
|---|---|---|
| (SEQ ID NOs:) | Fragment | Remarks |
| 13 and 14 | wcaJ upstream 1 | The sequences at the 5′ ends of the nucleotide |
| | | sequences represented by SEQ ID NOs: 3 and |
| | | 13 are complementary to each other. |
| 15 and 16 | wcaM downstream 1 | The sequences at the 5′ ends of the nucleotide |
| | | sequences represented by SEQ ID NOs: 6 and |
| | | 15 are complementary to each other. |
| 14 and 17 | wcaJ upstream 2 | The sequences at the 5′ ends of the nucleotide |
| 16 and 18 | wcaM downstream 2 | sequences represented by SEQ ID NOs: 17 |
| | | and 18 are complementary to each other. |

Here, wcaJ upstream 1 and wcaJ upstream 2 include the region from the start codon of wcaJ gene to about 900 bp upstream thereof. Moreover, wcaM downstream 1 and wcaM downstream 2 include the region from the stop codon of wcaM gene to about 800 bp downstream thereof.

PCR was performed using a mixture containing wcaJ upstream 1, wcaM downstream 1 and cat-sacB fragment at equivalent molar ratios as a template and using DNAs consisting of the nucleotide sequences represented by SEQ ID NOs:14 and 16 as a primer set, and a DNA fragment consisting of a sequence in which cat-sacB fragment was inserted into the sequence of a region around wcaJ-wzxC-wcaKLM operon (called wcaJ-wzxC-wcaKLM::cat-sacB below) was obtained.

PCR was performed using a mixture containing wcaJ upstream 2 and wcaM downstream 2 at equivalent molar ratios as a template and using DNAs consisting of the nucleotide sequences represented by SEQ ID NOs:14 and 16 as a primer set, and a DNA fragment consisting of a sequence which did not include wcaJ-wzxC-wcaKLM and in which the upstream of wcaJ and the downstream of wcaM were linked directly (called ΔwcaJ-wzxC-wcaKLMbelow) was obtained.

By introducing wcaJ-wzxC-wcaKLM::cat-sacB fragment into strain W3110ΔlacZY created above by the electroporation method, a transformant exhibiting resistance to chloramphenicol and sensitivity to sucrose (a transformant in which wcaJ-wzxC-wcaKLMwas replaced with wcaJ-wzxC-wcaKLM::cat-sacB) was obtained.

By introducing ΔwcaJM fragment into the transformant by the electroporation method, transformants exhibiting sensitivity to chloramphenicol and resistance to sucrose (transformants in which wcaJ-wzxC-wcaKLM::cat-sacB was replaced with ΔwcaJ-wzxC-wcaKLM) were obtained. A transformant exhibiting sensitivity to ampicillin (a transformant lacking pKD46) was further obtained. The transformant was named strain KFL.

(3) Preparation of Escherichia coli-Derived Lactose Permease (LacY) Expression Vector

PCR was performed using DNAs consisting of the nucleotide sequences shown in “Primer Set” in Table 4 as primer sets and the DNAs shown in “Template” in Table 4 as templates, and amplified DNA fragments were obtained.

TABLE 4

| Primer Set | | Amplified DNA |
|---|---|---|
| (SEQ ID NOs:) | Template | Fragment |
| 19 and 20 | Genomic DNA of Escherichia coli strain | lacY |
| | W3110 | |
| 21 and 22 | pHMFT (Japanese Patent No. 4048173) | HMFT |
| 23 and 24 | Genomic DNA of Escherichia coli strain | rcsA |
| | W3110 | |

The genomic DNA of Escherichia coli strain W3110 was prepared by a usual method. The nucleotide sequences represented by SEQ ID NOs:2 and 3 and those represented by SEQ ID NOs:4 and 5 each include complementary sequences at the 5′ ends.

PCR was performed using a mixture containing lacY, HMF T and resA fragments at equivalent molar ratios as a template and using DNAs consisting of the nucleotide sequences represented by SEQ ID NOs:25 and 26 as a primer set, and a DNA fragment in which the three fragments were linked (called lacY-HMFT-rcsA below) was obtained.

PCR was performed using DNAs consisting of the nucleotide sequences represented by SEQ ID NOs:27 and 28 as a primer set and using plasmid pPE167 (Appl. Environ. Microbiol. 2007, 73:6378-6385) as a template, and a vector fragment of about 4.4 kb was obtained. Here, the nucleotide sequences represented by SEQ ID NOs:25 and 28 and those represented by SEQ ID NOs:26 and 27 each include complementary sequences at the 5′ ends.

By linking lacY-THMFT-rcsA fragment and the vector fragment obtained above using In-Fusion HD Cloning Kit (manufactured by Takara Bio Inc.), expression plasmid pYHA1 was obtained.

### (4) Creation of Mutant LacY-Expressing Microorganism

Using plasmid pYHA1 obtained in (3) above as a template and using Prime STAR Mutagenesis Basal Kit (manufactured by Takara Bio Inc.), plasmid pYHA2, in which the L-lysine residue at position 319 of the amino acid sequence of LacY was replaced with L-glutamic acid residue, was created. DNAs consisting of the nucleotide sequences represented by SEQ ID NOs:29 and 30 were used as a primer set.

By transforming strain KFL created in (2) above using plasmid pYHA1 obtained in (3) and pYHA2, strain KFL/pYHA1 and strain KFL/pYHA2 were obtained.

### [Example 2] Production of 2′-Fucosyllactose by Fermentation Method Using Mutant LacY-Expressing Microorganism

Strain KFL/pYHA1 and strain KFL/pYHA2 obtained in Example 1 were cultured on a LB plate at 30° C. for 24 hours, inoculated to a large test tube containing 5 mL of LB medium containing 100 mg/L kanamycin and cultured by shaking at 30° C. for 16 hours. Then, 0.1 mL of the obtained culture solutions were inoculated to a large test tube containing 5 mL of a production medium containing 100 mg/L kanamycin [30 g/L glucose, 5 g/L lactose monohydrate, 2 g/L magnesium sulfate heptahydrate, 16 g/L dipotassium hydrogen phosphate, 14 g/L potassium dihydrogen phosphate, 2 g/L ammonium sulfate, 1 g/L citric acid monohydrate, 5 g/L casamino acid, 10 mg/L thiamine hydrochloride, 50 mg/L ferrous sulfate heptahydrate and 10 mg/L manganese sulfate pentahydrate (those except for glucose, lactose monohydrate and magnesium sulfate heptahydrate were adjusted to pH7.2 with an aqueous sodium hydroxide solution and then autoclaved) (aqueous solutions containing glucose, lactose monohydrate and magnesium sulfate heptahydrate were prepared separately, then autoclaved, each cooled and then mixed)] and cultured by shaking at 30° C. for 30 hours.

After the completion of the culturing, the culture solutions were appropriately diluted and then centrifuged, and 2′-fucosyllactose contained in the supernatants was analyzed by HPLC. The results are shown in Table 5.

TABLE 5

| Bacterial Strain Name | 2′-Fucosyllactose (g/L) |
|---|---|
| KFL/pYHA1 | 0.38 |
| KFL/pYHA2 | 2.58 |

As a result, strain KFL/pYHA2 exhibited a higher property of producing 2′-fucosyllactose than strain KFL/pYHA1.

It was found from the above results that, when a microorganism having mutant LacY is used, the property of producing 2′-fucosyllactose improves as compared to a microorganism having wild-type LacY.

The invention has been explained in detail referring to specific embodiments, but it is obvious to one skilled in the art that various changes and modifications are possible without departing from the spirit and the scope of the invention. The present application is based on a Japanese patent application filed on Dec. 16, 2019 (patent application No. 2019-226278), and the entire contents thereof are incorporated by reference. Moreover, all the references cited here are incorporated in their entirety.

### INDUSTRIAL APPLICABILITY

The invention provides a method for producing a lactose-containing oligosaccharide using a microorganism having an ability of producing a protein having modified lactose permease activity.

1. A microorganism which has a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3] below and
which has a higher ability of producing a lactose-containing oligosaccharide than a parent strain,
[1] a protein consisting of the amino acid sequence represented by SEQ ID NO:2,
[2] a mutant protein which is a protein consisting of an amino acid sequence having deletion, replacement, insertion and/or addition of 1 to 20 amino acids in the amino acid sequence represented by SEQ ID NO:2 and which has lactose permease activity, and
[3] a homologous protein which is a protein consisting of an amino acid sequence having at least 80% identity with the amino acid sequence represented by SEQ ID NO:2 and which has lactose permease activity.

2. The microorganism according to claim 1 which has a protein consisting of an amino acid sequence containing replacement of the amino acid residue corresponding to position 319 of the amino acid sequence represented by SEQ ID NO:2 with L-aspartic acid or L-glutamic acid in the amino acid sequence of the protein described in any one of [1] to [3].

3. The microorganism according to claim 1 which has a protein consisting of an amino acid sequence containing replacement of the amino acid residue corresponding to position 319 of the amino acid sequence represented by SEQ ID NO:2 with L-glutamic acid in the amino acid sequence of the protein described in any one of [1] to [3].

4. The microorganism according to claim 1 which is obtainable by transforming a parent strain with a recombinant DNA containing a DNA encoding a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3].

5. The microorganism according to claim 4 which is obtainable by incorporating the recombinant DNA containing a DNA encoding a protein consisting of an amino acid sequence containing replacement of the amino acid residue corresponding to position 319 of the amino acid sequence represented by SEQ ID NO:2 with a different amino acid residue in the amino acid sequence of the protein described in any one of [1] to [3] into the chromosome.

6. The microorganism according to claim 1 which is obtainable by transforming a parent strain with a recombinant DNA containing a DNA having the nucleotide sequence represented by SEQ ID NO:3.

7. The microorganism according to claim 6 which is obtainable by incorporating the recombinant DNA containing a DNA having the nucleotide sequence represented by SEQ ID NO:3 into the chromosome.

8. The microorganism according to claim 1, wherein the parent strain is a microorganism having an ability of producing a lactose-containing oligosaccharide.

9. A method for producing a lactose-containing oligosaccharide, comprising: culturing the microorganism according to claim 1 in a medium and producing a lactose-containing oligosaccharide in a culture.

10. The production method according to claim 9, wherein the lactose-containing oligosaccharide is 2′-fucosyllactose.


SEQUENCE LISTING (32 sequences)

SEQ ID NO: 1 - DNA, 1254 bp, Escherichia coli
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 2 - protein, 417 aa, Escherichia coli [sequence omitted]

SEQ ID NO: 3 - DNA, 1254 bp, Artificial Sequence
  Synthetic DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctggaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 4 - DNA, 20 bp, Artificial Sequence
  Synthetic DNA
accaggcgtttaagggcacc

SEQ ID NO: 5 - DNA, 45 bp, Artificial Sequence
  Synthetic DNA
gactacgggcctaaagtcgacagaataaataaatcctggtgtccc

SEQ ID NO: 6 - DNA, 28 bp, Artificial Sequence
  Synthetic DNA
gtcgactttaggcccgtagtctgcaaat

SEQ ID NO: 7 - DNA, 21 bp, Artificial Sequence
  Synthetic DNA
tacggttagccatttgcctgc

SEQ ID NO: 8 - DNA, 44 bp, Artificial Sequence
  Synthetic DNA
ggtgcccttaaacgcctggtagctgtttcctgtgtgaaattgtt

SEQ ID NO: 9 - DNA, 19 bp, Artificial Sequence
  Synthetic DNA
tcgcgcaacgcgtcagtgg

SEQ ID NO: 10 - DNA, 44 bp, Artificial Sequence
  Synthetic DNA
gcaggcaaatggctaaccgtaatgaacatgtcgatgacagaaag

SEQ ID NO: 11 - DNA, 20 bp, Artificial Sequence
  Synthetic DNA
tgcgctgtgtgtcgttgggc

SEQ ID NO: 12 - DNA, 45 bp, Artificial Sequence
  Synthetic DNA
ttctgtcatcgacatgttcatagctgtttcctgtgtgaaattgtt

SEQ ID NO: 13 - DNA, 23 bp, Artificial Sequence
  Synthetic DNA
atgaacatgtcgatgacagaaag

SEQ ID NO: 14 - DNA, 42 bp, Artificial Sequence
  Synthetic DNA
ggtgcccttaaacgcctggtcgttgttcctgttatttgcccc

SEQ ID NO: 15 - DNA, 21 bp, Artificial Sequence
  Synthetic DNA
aagctggtgattaactccggg

SEQ ID NO: 16 - DNA, 44 bp, Artificial Sequence
  Synthetic DNA
gcaggcaaatggctaaccgtaatttgcgaccattcctggaaaaa

SEQ ID NO: 17 - DNA, 20 bp, Artificial Sequence
  Synthetic DNA
gcacatagcgaccaacggcc

SEQ ID NO: 18 - DNA, 45 bp, Artificial Sequence
  Synthetic DNA
tttttccaggaatggtcgcaaatcgttgttcctgttatttgcccc

SEQ ID NO: 19 - DNA, 23 bp, Artificial Sequence
  Synthetic DNA
atttgcgaccattcctggaaaaa

SEQ ID NO: 20 - DNA, 28 bp, Artificial Sequence
  Synthetic DNA
atgtactatttaaaaaacacaaactttt

SEQ ID NO: 21 - DNA, 44 bp, Artificial Sequence
  Synthetic DNA
atccattctagacctccttaattaagcgacttcattcacctgac

SEQ ID NO: 22 - DNA, 39 bp, Artificial Sequence
  Synthetic DNA
ttaaggaggtctagaatggattttaagattgtgcaagtg

SEQ ID NO: 23 - DNA, 45 bp, Artificial Sequence
  Synthetic DNA
atggcataccctcactcaatgtcaagattttgtctcaaattggga

SEQ ID NO: 24 - DNA, 22 bp, Artificial Sequence
  Synthetic DNA
cattgagtgagggtatgccatg

SEQ ID NO: 25 - DNA, 24 bp, Artificial Sequence
  Synthetic DNA
ttagcgcatgttgacaaaaatacc

SEQ ID NO: 26 - DNA, 43 bp, Artificial Sequence
  Synthetic DNA
gaggagaaattaaccatgtactatttaaaaaacacaaactttt

SEQ ID NO: 27 - DNA, 39 bp, Artificial Sequence
  Synthetic DNA
agggcatcggtcgacttagcgcatgttgacaaaaatacc

SEQ ID NO: 28 - DNA, 21 bp, Artificial Sequence
  Synthetic DNA
gtcgaccgatgcccttgagag

SEQ ID NO: 29 - DNA, 25 bp, Artificial Sequence
  Synthetic DNA
ggttaatttctcctctttaatatcg

SEQ ID NO: 30 - DNA, 27 bp, Artificial Sequence
  Synthetic DNA
cagcgtttccagaataaccacttccag

SEQ ID NO: 31 - DNA, 27 bp, Artificial Sequence
  Synthetic DNA
attctggaaacgctgcatatgtttgaa

SEQ ID NO: 32 - protein, 417 aa, Artificial Sequence
  Artificial Protein
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEFGRARMFGCVGWALCASIVGIMFTINNQFVFWLGSGCALILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILETLHMFEVPFLLVGCFKYITSQFEVRFSATIYLVCFCFFKQLAMIFMSVLAGNMYESIGFQGAYLVLGLVALGFTLISVFTLSGPGPLSLLRRQVNEVA